(19) United States
(12) Patent Application Publication
(10) Pub. No.: US 2026/0259172 A1
(43) Pub. Date: Sep. 3, 2026

(54) METHOD FOR IDENTIFYING OPTICAL ISOMER OF LOW-MOLECULAR-WEIGHT COMPOUND

(71) Applicant: The University of Osaka, Osaka (JP)

(72) Inventors: Masateru TANIGUCHI, Osaka (JP); Takahito OHSHIRO, Osaka (JP)

(21) Appl. No.: 19/165,104

(22) PCT Filed: Mar. 11, 2024
(86) PCT No.: PCT/JP2024/009238

§ 371 Date: Sep. 12, 2025

(30) Foreign Application Priority Data
Mar. 13, 2023 (JP) 2023-039102

(51) Int. Cl. G01N 27/49; G01N 27/30; G01N 27/447; G01N 33/68

(52) U.S. Cl. CPC G01N 27/49; G01N 27/30; G01N 27/447; G01N 33/6812

(57) ABSTRACT

The present invention addresses the problem of providing a method for identifying an optical isomer of a low-molecular-weight compound, the method making it possible to analyze whether the optical isomer is in a D-form or an L-form for each measured low-molecular-weight compound. This method for identification comprises: a low-molecular-weight compound electrophoresis step which is performed using a device including measurement electrodes for measuring a tunnel current at the time when a low-molecular-weight compound passes therethrough, and in which a voltage is applied so as to straddle the measurement electrodes of the device, thereby allowing the low-molecular-weight compound contained in a sample solution to pass through the measurement electrodes by electrophoresis; a measurement step in which a tunnel current at the time when the low-molecular-weight compound passes through a gap between the measurement electrodes is measured; and an analysis step in which it is determined, from the measured tunnel current, whether an optical isomer is in a D-form or an L-form for every measured low-molecular-weight compound.

### TECHNICAL FIELD

The present disclosure of the present application relates to an identifying method of optical isomers of a low-molecular compound.

### BACKGROUND ART

In low-molecular compounds such as sugars and amino acids, there are optical isomers consisting of a D-isomer and an L-isomer that are in an enantiomeric relationship with each other. In the fields of biochemistry, natural product chemistry, pharmacology, or the like, it is important to discriminate optical isomers of compounds from each other. While one of the enantiomers of the optical isomers has pharmacological activity, there are not a few examples in which the other of the enantiomers functions as a poison, as seen in a thalidomide disaster, for example. Thus, in particular, in the fields of medicines, pharmaceutical development, foods, or the like, there is a demand for a scheme to identify optical isomers (D-isomer, L-isomer) of a low-molecular compound in a simple manner or calculate the ratio of optical isomers.

As identifying methods of optical isomers, optical measurement method such as circular dichroism (CD) measurement or optical rotation measurement using optical absorption spectrum, methods of analyzing optical isomers using ion mobility spectrometry (IMS) method, and the like are known.

However, the optical measurement methods described above have problems of longer analysis time, need for a certain amount of a sample, and difficulty in application when the quantity of the sample is significantly small.

In the IMS method described above, when molecular ions produced from compounds contained in a sample are moved in a medium gas (or liquid) by the effect of an electric field, the ions are moved at a speed in accordance with mobility determined by the collision sectional area, which depends on the molecular size or the like, or the electric field intensity. The IMS method is a measurement method using this mobility for analysis of sample molecules. However, since a D-isomer and an L-isomer, which are optical isomers, have structures in a mirror image relationship and have the same mass and size, there is no difference in the collision sectional area. Thus, a chiral gas having the structure similar to a compound to be detected is mixed to a gas flowing through a region where ions are drifted in the IMS method. In response, interaction depending on chiral symmetry occurs between the molecular ion of the compound to be detected and the chiral gas, and such interaction changes the effective collision sectional area of molecular ions of the compound to be detected. In the IMS method, by utilizing such a property, it is possible to isolate and detect the optical isomers. In the method described above, however, it is required to prepare a chiral gas corresponding to a compound to be detected. Thus, while there is no problem when only a particular compound is to be detected, when various compounds are intended to be detected, it is necessary to prepare multiple types of chiral gases corresponding to these compounds, which increases the cost of measurement.

Further, it is not always possible to prepare a suitable chiral gas for a particular compound, and in such a case, there is a problem of inability of using the analysis scheme described above.

To solve the problems described above, Patent Literature 1 discloses that, when identifying optical isomers of a target compound by using an ion mobility analysis device, it is possible to identify whether the target compound is of a D-isomer or an L-isomer by having:
a) a light irradiation step of irradiating ions with left-handed or right-handed circularly polarized light, in which the ions are derived from the target compound introduced into the drift region or being drifting in the drift region;
b) a measurement step of measuring the relationship between the drift time and the ion intensity of the ions when the ions are irradiated with the left-handed or right-handed circularly polarized light; and
c) an optical isomer analysis step of, based on a measurement result obtained in the measurement step, identifying the optical isomers of the target compound or estimating the existence ratio of the optical isomers.

### CITATION LIST

### Patent Literature

Patent Literature 1: Japanese Patent Application Laid-Open No. 2019-39698

### SUMMARY OF INVENTION

### Technical Problem

As described above, methods of identifying optical isomers are known. In the method disclosed in Patent Literature 1 described above, however, it is required to ionize a sample, and there are problems of a still long measurement time and a large and expensive device. Thus, there is a demand for development of a method that can identify an optical isomer of a low-molecular compound by using a novel principle.

The disclosure in the present application has been made to solve the problems described above. As a result of intensive studies, the present inventors have newly found that (1) by measuring a low-molecular compound by using a device that can measure tunnel current (2) it is possible to identify whether each measured low-molecular compound is of the D-isomer or the L-isomer.

The goal of the disclosure of the present application is to provide an identifying method of optical isomers of a low-molecular compound that can analyze whether each measured low-molecular compound is of the D-isomer or the L-isomer.

### Solution to Problem

The disclosure of the present application relates to an identifying method of optical isomers of a low-molecular compound, as illustrated below.
(1) An identifying method of optical isomers of a low-molecular compound, wherein the identifying method is performed by using a device including measuring electrodes for measuring tunnel current occurring when the low-molecular compound passes between the measuring electrodes, the identifying method including:
   a low-molecular compound electrophoresis step of applying a voltage so as to span the measuring electrodes of the device to cause a low-molecular compound contained in a sample solution to pass between the measuring electrodes by electrophoresis;
   a measurement step of measuring tunnel current occurring when the low-molecular compound passes through a gap between the measuring electrodes; and
   an analysis step of analyzing from the measured tunnel current whether each measured low-molecular compound is of a D-isomer or an L-isomer.
(2) The identifying method according to (1) above further including, subsequent to the analysis step, a ratio calculation step of calculating a ratio of D-isomers and L-isomers of the low-molecular compound contained in the sample solution.
(3) The identifying method according to (1) or (2) above, wherein the low-molecular compound is an amino acid, and the amino acid is of one or more types selected from a group consisting of alanine, arginine, asparagine, aspartic acid, cysteine, glutamine, glutamic acid, histidine, isoleucine, leucine, lysine, methionine, phenylalanine, proline, serine, threonine, tryptophan, tyrosine, and valine.
(4) The identifying method according to (1) or (2) above, wherein the measuring electrodes are not modified with a substance that interacts with the low-molecular compound.
(5) The identifying method according to (3) above, wherein the measuring electrodes are not modified with a substance that interacts with the low-molecular compound.

### Advantageous Effects of Invention

The use of the identifying method of optical isomers of a low-molecular compound disclosed in the present application makes it possible to identify whether each measured low-molecular compound is of the D-isomer or the L-isomer.

### BRIEF DESCRIPTION OF DRAWINGS

FIG. 1A is a top view illustrating an overview of a device 1.

FIG. 1B is an arrow X-X sectional view of FIG. 1A.

FIG. 1C is an arrow Y-Y sectional view of FIG. 1A.

FIG. 2A is a flowchart illustrating an example of an identifying method of optical isomers.

FIG. 2B is a diagram illustrating an example of a feature amount in machine learning.

FIG. 3 is a schematic diagram illustrating an example of a configuration including the device 1 when the identifying method of optical isomers is performed.

FIG. 4 represents photographs substituted for a drawing, which represents a photograph of the produced device 1 and an enlarged photograph of a part near the measurement channel 32 in which measuring electrodes 4 are arranged.

FIG. 5A represents an analysis result obtained when L-isomer tryptophan and D-isomer tryptophan are used as amino acids and measured separately.

FIG. 5B represents a confusion matrix illustrating the analysis result obtained when L-isomer tryptophan and D-isomer tryptophan are used as amino acids and measured separately.

FIG. 6 illustrates results obtained when the L-isomer and the D-isomer in an amino acid mixed solution of Example 2 are identified.

FIG. 7 illustrates results obtained when the L-isomer and the D-isomer in an amino acid mixed solution of Example 3 are identified.

FIG. 8 illustrates waveform signals of four types of alcohol molecules measured in Example 5.

FIG. 9 illustrates results obtained when the L-isomer and the D-isomer of four types of alcohol molecules measured in Example 5 are identified.

FIG. 10 illustrates results obtained when the L-isomer and the D-isomer in nucleic acid and amino acid mixed solutions measured in Example 6 are identified.

FIG. 11 illustrates quantified results for an amino acid mixed solution measured in Example 7.

FIG. 12 illustrates a quantified result for an amino acid mixed solution measured in Example 8.

### DESCRIPTION OF EMBODIMENTS

An identifying method of optical isomers of a low-molecular compound will be described below in detail with reference to the drawings.

In this specification, members having the same type of function are labeled with the same or similar reference symbols. Further, repeated description for the members labeled with the same or similar reference symbols may be omitted.

In this specification, a numerical range expressed by using “to” means a range including numerical values preceding and subsequent to “to” as the lower limit and the upper limit, respectively. A numerical value, a numerical range, and a qualitative expression (for example, an expression of “the same”, “substantially”, or the like) is to be construed as indicating a numerical value, a numerical range, and a nature including an error generally tolerated in the field of the art.

Further, the position, the size, the range, or the like of each component illustrated in the drawings may not necessarily represent an actual position, an actual size, an actual range, or the like for easier understanding. Thus, the disclosure of the present application is not necessarily limited to the position, the size, the range, or the like disclosed in the drawings.

### Embodiment of Identifying Method of Optical Isomers of Low-Molecular Compound

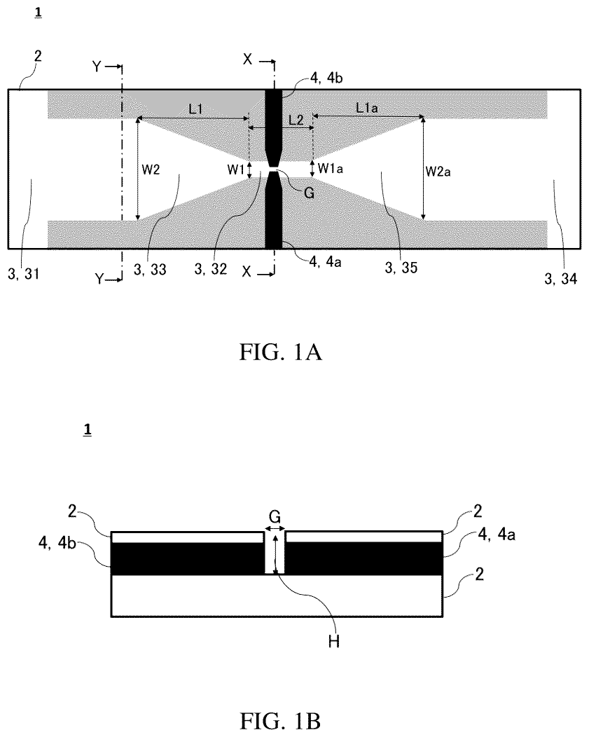

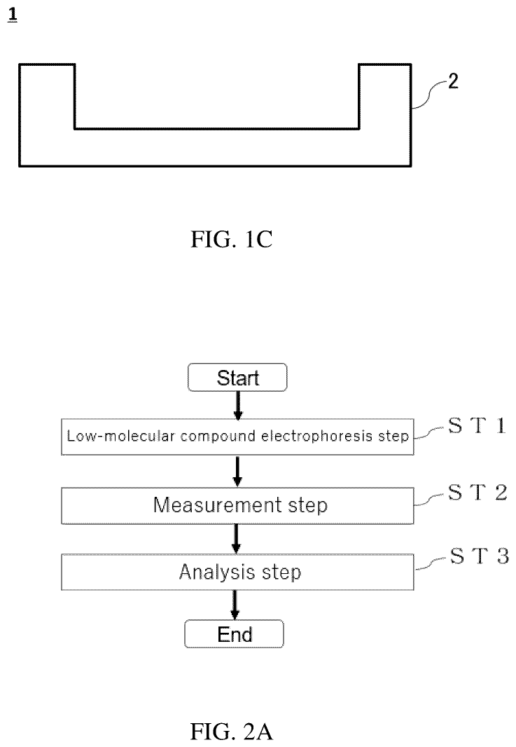

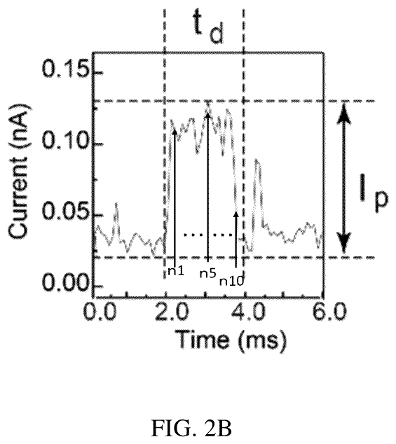

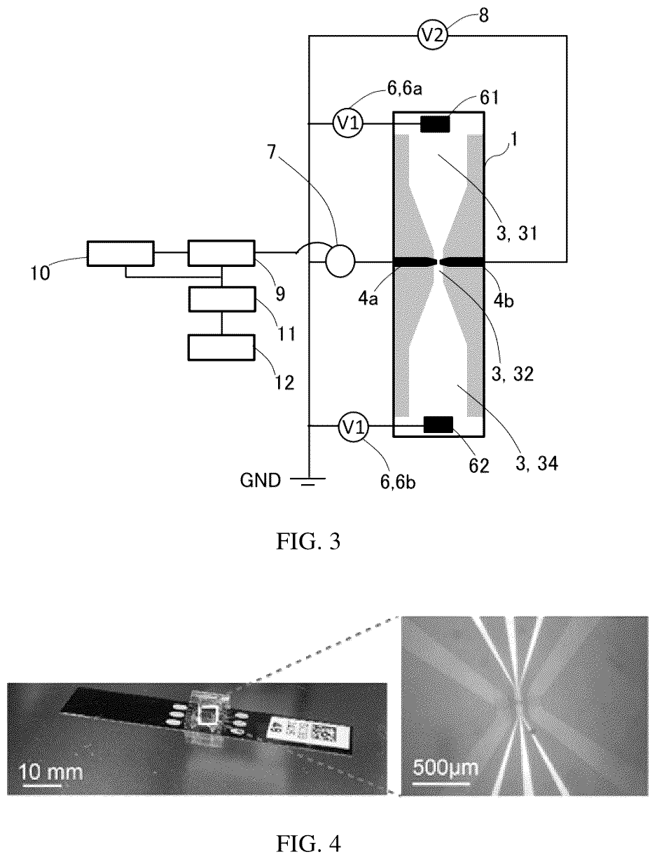

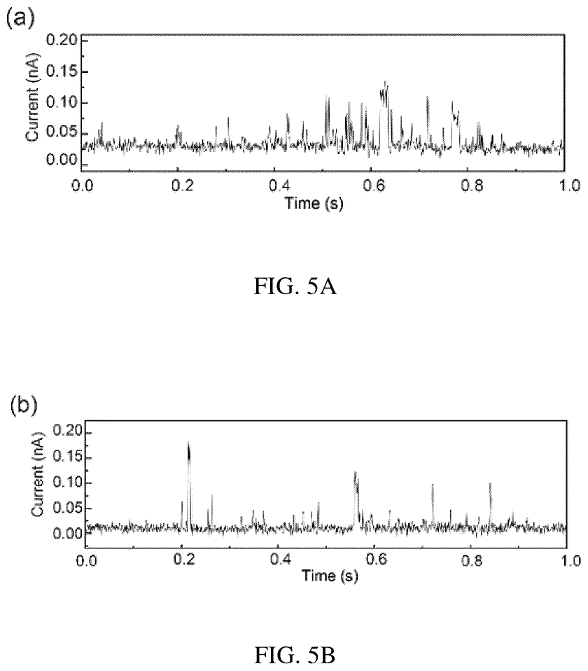

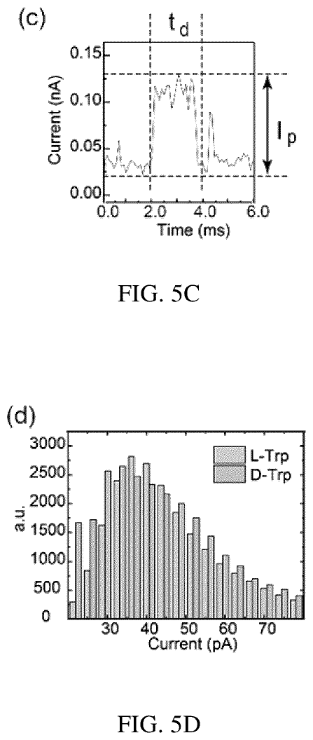

An embodiment of an identifying method of optical isomers of a low-molecular compound (hereafter, which may be simply referred to as “identifying method”) will be described with reference to FIG. 1 to FIG. 3. FIG. 1A to FIG. 1C are diagrams illustrating an example of a device used in the embodiment of the identifying method, FIG. 1A is a top view illustrating an overview of the device 1, FIG. 1B is an arrow X-X sectional view of FIG. 1A, and FIG. 1C is an arrow Y-Y sectional view of FIG. 1A. FIG. 2A is a flowchart illustrating an example of the identifying method. FIG. 2B is a diagram illustrating an example of a feature amount in machine learning. FIG. 3 is a schematic diagram illustrating an example of the configuration including the device 1 when the identifying method of optical isomers is performed.

The identifying method of optical isomers is performed by using a device that can measure tunnel current occurring when a low-molecular compound passes therethrough. The device includes a measurement unit for measuring tunnel current occurring when a low-molecular compound passes therethrough, and the measurement unit is not particularly limited as long as it can measure tunnel current occurring when a low-molecular compound passes through a gap between measuring electrodes arranged in the measurement unit. An example of the device 1 used in the identifying method of optical isomers will be described with reference to FIG. 1A to FIG. 1C and FIG. 3.

In the example illustrated in FIG. 1A to FIG. 1C and FIG. 3, the device 1 includes a substrate 2, a channel 3 formed in the substrate 2, and a pair of measuring electrodes 4 (4a and 4b) used for measuring tunnel current occurring when a low-molecular compound passes therebetween. The channel 3 includes a sample solution supply channel 31 into which a sample solution containing a low-molecular compound is supplied, a measurement channel 32 in which the measuring electrodes 4 are arranged, a first tapered channel 33 arranged between the sample solution supply channel 31 and the measurement channel 32 and having a channel width decreasing from the sample solution supply channel 31 to the measurement channel 32, and a collection channel 34 that collects a low-molecular compound that has passed through the measurement channel 32.

Although a second tapered channel 35 is depicted in the example illustrated in FIG. 1A, the second tapered channel 35 has an optional, additional configuration. The collection channel 34 may be coupled directly to the measurement channel 32 as long as a low-molecular compound flowing out of the measurement channel 32 can be collected.

For example, the device 1 can be manufactured using a nanochannel-integrated mechanically controllable break junction. Note that a mechanically controllable break junction (MCBJ) for producing the pair of measuring electrodes 4 is disclosed by Japanese Patent Application Laid-Open No. 2019-525766; M. Tsutsui, K., Shoji, M. Taniguchi, T. Kawai, Nano Lett., 345 (2008); M. Tsutsui, M. Taniguchi, T. Kawai, Appl. Phys. Lett. 93, 163115 (2008), and the like, for example. Therefore, detailed description for the method of manufacturing the device 1 will be omitted.

The substrate 2 is not particularly limited as long as it is a material generally used in the field of semiconductor manufacturing technologies. The material of the substrate 2 may be, for example, Si, SiOₓ, SiNₓ, Ge, Se, Te, GaAs, GaP, GaN, InSb, InP, or the like.

The material used for forming the measuring electrodes 4 is not particularly limited as long as it can be used for measuring tunnel current. For example, the material may be gold, platinum, silver, palladium, tungsten, an alloy of these metals, or the like. Note that, while relying on a different principle from the principle of the identifying method disclosed in the present application (which is to measure “tunnel current” occurring when a low-molecular compound passes between the measuring electrodes 4), International Publication No. 2017/183716 discloses a technical concept that a substance that interacts with a measurement target is provided to nanopores, and thereby the accuracy in identifying the measurement target is improved. In the identifying method according to the embodiment, however, it is not required to modify the measuring electrodes 4 with a substance that interacts with D-isomers or L-isomers or the like, and it is possible to identify a D-isomer or an L-isomer by using produced measuring electrodes 4 without any change.

When a voltage is applied to the sample solution supply channel 31 and the collection channel 34, electrophoretic force is applied to a low-molecular compound, and the moving speed of the low-molecular compound is increased. As a result, compared to a case where no electrophoretic force is applied, the measurement speed for the low-molecular compound is improved. In contrast, when a voltage is applied to the channel 3 to apply electrophoretic force to a low-molecular compound, a larger sectional area of the channel 3 will require a larger voltage.

The measurement on a low-molecular compound by using tunnel current is performed by identifying the difference in the measured current value at a picoampere level. When a voltage at such a level that can apply electrophoretic force is applied to a low-molecular compound supplied into a channel of micrometer order, the measuring electrodes 4 may undesirably detect noise caused by the voltage for electrophoresis. The device 1 illustrated in FIG. 1A to FIG. 1C can apply electrophoretic force to a sample at a low voltage and thus can achieve measurement of tunnel current with less noise that would otherwise be caused by the voltage for electrophoresis.

Since a sample solution is supplied into the device 1, a predetermined size of the sample solution supply channel 31 is required. Thus, the device 1 employes the structure having a narrowed (reduced) width of the measurement channel 32 where the measuring electrodes 4 are arranged and connecting the sample solution supply channel 31 and the measurement channel 32 via the first tapered channel 33.

As described above, to reduce noise due to a voltage applied for electrophoresis, it is preferable that the width of the measurement channel 32 be narrower. When the width of the connection part between the first taper channel 33 and the measurement channel 32 is denoted as W1, W1 can be 200 nm or less, 180 nm or less, 160 nm or less, 140 nm or less, 120 nm or less, 100 nm or less, 90 nm or less, 80 nm or less, 70 nm or less, 60 nm or less, or 50 nm or less. On the other hand, there is no limitation in the lower limit of W1 as long as it is within a manufacturable range, and the lower limit of W1 can be, but is not limited to, 20 nm or greater, 25 nm or greater, or 30 nm or greater, for example.

The width of the measurement channel 32 may be the same along the entire length or may vary along the length as long as it is within a range that does not affect analysis of a measurement result or the like. In the example illustrated in FIG. 1A, when the end opposite to the width W1 of the measurement channel 32 is denoted as W1a, W1a may be the same as W1 or may be larger or smaller than W1.

The gap between the pair of measuring electrodes 4a and 4b (the gap G, see FIG. 1B) is not particularly limited as long as the tunnel current occurring when a low-molecular compound passes therethrough can be measured. The gap G can be, for example, but is not limited to, 0.1 nm or larger, 0.3 nm or greater, 0.5 nm or greater, 0.7 nm or greater, or 0.9 nm or greater. On the other hand, the upper limit of the gap G can be, for example, but is not limited to, 50 nm or less, 30 nm or less, 10 nm or less, 8 nm or less, 6 nm or less, 5 nm or less, 4 nm or less, 3 nm or less, 2 nm or less, or 1 nm or less. Note that the gap G between the measuring electrodes 4a and 4b affects the sensitivity of identification. Since it is difficult to have completely the same gap G for a plurality of produced devices 1, it is desirable to produce the gap G on a device basis when data of a known low-molecular compound described later is collected.

The length of the measuring electrode 4 (the length of the gap G in the same direction as L2 of FIG. 1A) is also not particularly limited as long as it is within a range that enables measurement of tunnel current occurring when a low-molecular compound passes therebetween. The length can be, but is not limited to, 1000 nm or less, 800 nm or less, 600 nm or less, 400 nm or less, 200 nm or less, 100 nm or less, 80 nm or less, or 60 nm or less, for example.

Note that, for easier cutting in MCBJ, a smaller deposition amount of the measuring electrodes 4 (in a direction orthogonal to the direction of the length of the measuring electrode 4 or in the direction H in FIG. 1B, hereafter, which may be denoted as “thickness”) is preferable. An increase in the thickness of the measuring electrode 4 may make it difficult to control a cutting place and result in a rough cut surface of the fabricated gap G. Thus, the thickness of the measuring electrode 4 can be, but is not limited to, 80 nm or less, 70 nm or less, 60 nm or less, or 50 nm or less, for example. The lower limit of the thickness of the measuring electrode 4 is not particularly limited as long as tunnel current can be measured and can be, but is not limited to, 2 nm or greater, 4 nm or greater, 6 nm or greater, 8 nm or greater, 10 nm or greater, 15 nm or greater, or 20 nm or greater, for example.

As described above, it is preferable that the length of the measuring electrode 4 be larger than the thickness thereof in order to reduce the thickness of the measuring electrode 4 to form the gap G by MCBJ. The ratio of length/thickness may be, but is not limited to, 10 to 100, for example.

The length L2 of the measurement channel 32 is not particularly limited as long as it is within a range that enables measurement of tunnel current occurring when a low-molecular compound passes therethrough. If the length L2 is too long, the entire channel of the device 1 will be longer. In contrast, if the length L2 is too short, it will be difficult to maintain an elongated state of a low-molecular compound. The length L2 can be, but is not limited to, 20 nm or greater, 25 nm or greater, 30 nm or greater, 35 nm or greater, 40 nm or greater, 45 nm or greater, or 50 nm or greater. Further, the length L2 can be 2000 nm or less, 1500 nm or less, 1000 nm or less, 800 nm or less, 600 nm or less, 400 nm or less, 200 nm or less, 180 nm or less, 160 nm or less, 140 nm or less, 120 nm or less or 100 nm or less. The length L2 is naturally required to be longer than the length of the gap G part of the measuring electrodes 4.

To reduce noise due to a voltage applied for electrophoresis, it is preferable that the depth H of the channel 3 be also smaller. The depth H of the channel 3 can be, but is not limited to, 200 nm or less, 180 nm or less, 160 nm or less, 140 nm or less, 120 nm or less, 100 nm or less, 90 nm or less, 80 nm or less, 70 nm or less, 60 nm or less, or 50 nm or less, for example. On the other hand, the depth H of the channel 3 can be 20 nm or greater, 25 nm or greater, or 30 nm or greater, for example.

In the device 1 illustrated in FIG. 1A to FIG. 1C, although the length of the first tapered channel 33 (L1 in FIG. 1A) and the width of the sample solution supply channel 31 (W2 in FIG. 1A, a portion connected to the first tapered channel 33) are not particularly limited, it is desirable to reduce the width of the channel 3 as much as possible. Note that, to supply a sample solution, the sample solution supply channel 31 may have a wider portion having a width larger than W2 as needed.

In device 1, although the width of the collection channel 34 and the length of the optionally, additionally provided second tapered channel 35 (L1a in FIG. 1A) are not particularly limited, it is desirable to reduce the width of the channel 3 as much as possible. Note that, to collect a low-molecular compound, the collection channel 34 may have a wider portion having a width larger than W2a as needed.

In the device 1, when the width of the connection part between the first taper channel 33 and the measurement channel 32 is denoted as W1, and the width of the connection part between the first taper channel 33 and the sample solution supply channel 31 is denoted as W2, the lower limit of W2/W1 may be 2 or greater, 3 or greater, 4 or greater, 5 or greater, 6 or greater, 7 or greater, 8 or greater, 9 or greater, or 10 or greater, and the upper limit of W2/W1 may be 50 or less, 40 or less, 30 or less, or 20 or less. Further, when the length between the connection part between the first taper channel 33 and the measurement channel 32 and the connection part between the first taper channel and the sample solution supply channel is denoted as L1, the lower limit of L1/W2 may be 0.3 or greater, 0.4 or greater, or 0.5 or greater, and the upper limit of L1/W2 may be 10 or less, 9 or less, 8 or less, 7 or less, 6 or less, or 5 or less.

The device 1 may include the second tapered channel 35 arranged between the measurement channel 32 and the collection channel 34 and having a channel width that increases from the measurement channel 32 to the collection channel 34.

Next, the identifying method will be described in detail with reference to FIG. 2A, FIG. 2B, and FIG. 3. The identifying method includes a low-molecular compound electrophoresis step (ST1), a measurement step (ST2), and an analysis step (ST3).

In the low-molecular compound electrophoresis step (ST1), a voltage is applied so as to span the measuring electrodes 4a and 4b of the device 1, and thereby a low-molecular compound contained in a sample solution passes between the measuring electrodes 4a and 4b by electrophoresis.

The sample solution is not particularly limited as long as it contains a low-molecular compound. Note that, in the present specification, “low-molecular compound” means a compound whose molecular weight is less than or equal to 10000. Further, optical isomers including D-isomers and L-isomers are not particularly limited as long as they are compounds having the chiral center (asymmetric atoms such as carbon, silicon, germanium, lead, or the like). Examples of compounds having the chiral center may be an organic compound or the like having asymmetric carbon atoms, such as amino acids, sugars, lipids, lactic acids, or the like.

The number of asymmetric atoms of an optical isomer is not limited to one and may be two or greater or three or greater. When two or more asymmetric atoms are included, the asymmetric atoms may be the same or may be different from each other.

Proteins of humans are made of 20 types of amino acids, more specifically, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. Among these 20 types of amino acids, glycine has no asymmetric carbon atom and thus has neither D-isomer nor L-isomer. In contrast, the remaining 19 types of amino acids other than glycine have asymmetric carbon atoms and thus have D-isomers and L-isomers. The use of the identifying method disclosed in the present application makes it possible to identify whether the 19 types of amino acids listed above contained in a sample solution are of the D-isomer or the L-isomer.

Note that, although the 19 types of amino acids listed above are described as examples that include both D-isomers and L-isomers, the low-molecular compound disclosed in the present application may be any low-molecular compound whose molecular weight is less than or equal to 10000, and it is only necessary for the optical isomer to have one or more asymmetric atoms. Therefore, peptides having two or more multiple amino acids coupled in a chain shape by peptide bond are also included in the low-molecular compound in the present application. Further, when viewed in terms of peptides, since it is only necessary that an asymmetric carbon atom is included in any of the amino acids that make up a peptide, glycine may be included in peptides.

It is only required for a solvent used for producing a sample solution to be electroconductive. The solvent may be, for example, but is not limited to, ultrapure water, buffer solutions, or the like. For example, the ultrapure water can be manufactured by using Milli-Q (registered trademark) Integral3 (device name) manufactured by EMD Millipore (Milli-Q (registered trademark) Integral 33/5/1015 (catalog number)). The buffer solution may be a known buffer for electrophoresis, such as a TE buffer, a TBE buffer, a PBS buffer, or the like. The concentration of the buffer can be adjusted as appropriate within a range that enables electrophoresis, such as, for example, but is not limited to, 1 μM or less. In the first tapered channel 33, a measurement channel 32, and the second tapered channel 35, which is formed as needed, liquid junction is ensured by permeation of a supplied sample solution or solvent by capillary force.

In the example illustrated in FIG. 3, a measurement unit is formed of the measurement channel 32 and the pair of measuring electrodes 4a and 4b arranged in the measurement channel 32. Further, an electrophoresis first electrode (hereafter, which may be referred to as “first electrode”) 61 is formed at a location within the sample solution supply channel 31 and in contact with a sample solution, and an electrophoresis second electrode (hereafter, which may be referred to as “second electrode”) 62 is formed at a location within the collection channel 34 and in contact with a solvent. Further, a first power supply 6a connected to the first electrode 61 and a first power supply 6b connected to the second electrode 62 are used to apply a voltage so as to span the measuring electrodes 4a and 4b, and thereby the low-molecular compound passes between the measuring electrodes 4a and 4b by electrophoresis. Note that, although FIG. 3 illustrates an example of using two first power supplies 6 to apply a voltage for electrophoresis, a single first power supply 6 may be used.

The first electrode 61 and the second electrode 62 can be formed of a known electroconductive metal such as Ag/AgCl, aluminum, copper, platinum, gold, silver, or titanium. The first electrode 61 and the second electrode 62 may be formed on the substrate 2 or may be a separate member from the device 1 and inserted via a hole in a cover member (not illustrated).

When the voltage applied by the first power supply 6 is excessively small, the moving speed of a low-molecular compound will be lower, and the time required for measurement will be longer. This voltage may be, for example, but is not limited to, 10 mV or greater, 15 mV or greater, 20 mV or greater, 25 mV or greater, or 30 mV or greater. On the other hand, the upper limit of the voltage applied by the first power supply 6 can be set as appropriate taking the accuracy of the analysis step described later, the width of the channel 3, and the like into consideration. This upper limit may be, for example, but is not limited to, 5 V or less, 3 V or less, 1 V or less, 500 mV or less, 300 mV or less, 100 mV or less, 90 mV or less, 80 mV or less, 70 mV or less, 60 mV or less, or 50 mV or less.

In the measurement step (ST2), tunnel current occurring when a low-molecular compound passes through the gap between the measuring electrodes 4a and 4b is measured. In the example illustrated in FIG. 3, the measurement step (ST2) is to apply a voltage across the pair of measuring electrodes 4a and 4b by using a power supply for tunnel current measurement (hereafter, which may be referred to as “second power supply”) 8 and measure a change in tunnel current occurring when the low-molecular compound passes through the gap between the pair of measuring electrodes 4a and 4b by using a tunnel current detection unit 7. Note that the example illustrated in FIG. 3 is an example of the measurement step (ST2) and is not limited thereto. Since a change in the generated tunnel current is in a picoampere level, a known ammeter that can measure current at a picoampere level can be used for the tunnel current detection unit 7. Further, the current value may be calculated from a voltage measured by a voltmeter. The tunnel current detection unit 7 may optionally, additionally include a current amplifier, a noise removal device, an analog-to-digital (A/D) converter, or the like. When the tunnel current detection unit 7 includes a current amplifier, a noise removal device, an A/D converter, or the like, it is possible to provide easily analyzable data instead of raw data of the measured tunnel current value. Alternatively, the tunnel current detection unit 7 may be configured to measure only a change in the tunnel current, and the current amplifier, the noise removal device, the A/D converter, or the like may be of configurations in an analysis unit 9.

The second power supply 8 applies a voltage across the pair of measuring electrodes 4a and 4b. The voltage applied by the second power supply 8 is not particularly limited as long as tunnel current can be measured. Although not limited, for example, the lower limit of the voltage may be 20 mV or greater, 50 mV or greater, or 100 mV or greater, and the upper limit of the voltage may be 750 mV or less, 500 mV or less, 250 mV or less, or the like. The specific configuration of the second power supply 8 is not particularly limited, and a known power supply device can be used. In the example illustrated in FIG. 3, when the device 1 has a significantly reduced width of the channel 3, in particular, the measurement channel 32, this can reduce the voltage required for electrophoresis of a low-molecular compound. Thus, in the tunnel current detection unit 7, a measurement value of tunnel current with a small noise component is obtained in measurement of tunnel current occurring when the low-molecular compound passes through the gap between the measuring electrodes 4.

The analysis step (ST3) is to analyze whether each measured low-molecular compound is of the D-isomer or the L-isomer from a measurement result of the tunnel current measured in the measurement step. In the example illustrated in FIG. 3, the analysis step (ST3) is performed in the analysis unit 9 (hereafter, the analysis step performed in the analysis unit may be simply referred to as “analysis unit”). As an example of the analysis step (ST3), the analysis unit 9 calculates the conductance from the measurement value of the tunnel current. The conductance can be calculated by dividing a measurement value of tunnel current by a voltage applied across the pair of measuring electrodes 4a and 4b. The conductance calculated from tunnel current occurring when the low-molecular compound passes between the pair of measuring electrodes 4a and 4b differs in accordance with the type of the low-molecular compound and, even in the case of the same type, differs in accordance with whether the low-molecular compound is of a D-isomer or an L-isomer. It is therefore possible to determine the type of a low-molecular compound and determine whether the low-molecular compound is of a D-isomer or an L-isomer by comparing a measured conductance of the low-molecular compound with the conductance of the D-isomer and the L-isomer of each known low-molecular compound measured and calculated in advance.

In the analysis step (ST3), analysis may be performed by machine learning as needed. The analysis accuracy can be improved by performing machine learning on waveform signals of tunnel current obtained when the low-molecular compound passes between the pair of measuring electrodes 4a and 4b. For a classification analysis scheme with machine learning, a known scheme may be used. For example, the scheme may be a random forest method, k-nearest neighbors, a Naïve Bayes classifier, a decision tree, a neural network (a convolutional neural network, a recurrent neural network, or the like), a support vector machine, a bagging method, a boosting method, clustering (hierarchical clustering, DBSCAN, or the like)), dimensionality reduction (Principal Component Analysis (PCA), t-SNE, LLE, or the like), anomaly detection (Local Outlier Factor (LOF), isolation forest, or the like) self-organizing map, a generative model, an autoencoder, or the like.

When analysis is performed by machine learning, a classification analysis program that performs classification analysis with machine learning can be stored in a program memory 11 described later. The analysis unit 9 then finds in advance feature amounts representing features of waveform signals obtained from known low-molecular compounds and uses the previously found feature amount as training data for machine learning. Then, the feature amount of the waveform signal obtained from a low-molecular compound to be measured can be used as a variable to perform the classification analysis program to analyze the type of the low-molecular compound to be measured and analyze whether the low-molecular compound is of a D-isomer or an L-isomer. Note that the detailed procedure of the machine learning is disclosed in Japanese Patent Application Laid-Open No. 2020-173259, International Publication No. 2020/017608, International Publication No. 2018/207524, Japanese Patent Application Laid-Open No. 2017-120257, and the like. The procedure disclosed in the above publications can apply for implementation of the embodiment. The features disclosed in Japanese Patent Application Laid-Open No. 2020-173259, International Publication No. 2020/017608, International Publication No. 2018/207524, and Japanese Patent Application Laid-Open No. 2017-120257 are incorporated in the present specification by reference.

Note that, in performing machine learning, the present inventors have intensively examined a preferable feature amount in terms of identifying whether a low-molecular compound is of a D-isomer or an L-isomer based on a waveform signal of tunnel current occurring when the low-molecular compound passes between the pair of measuring electrodes 4a and 4b. As a result, it has been found that a low-molecular compound to be measured can be accurately identified when a standardized amount of a time averaged value of signal n-division is used as the feature amount. The newly found feature amount will be described with reference to FIG. 2B. First, the signal means a change in tunnel current occurring when the low-molecular compound passes through the gap between the measuring electrodes 4. In the example illustrated in FIG. 2B, a change in tunnel current during td corresponds to a signal. The signal n-division means dividing td into n. While the value n is not particularly limited as long as it is an integer, a smaller number of divisions results in poor accuracy in identification. For example, the number of divisions can be suitably set to 3 or greater, 4 or greater, 5 or greater, 6 or greater, 7 or greater, 8 or greater, 9 or greater, 10 or greater, 11 or greater, 12 or greater, or the like. On the other hand, since the value n depends on a sampling rate, the upper limit thereof can be about 1000 or less. The standardization of a time averaged value of signal n-division means standardizing an averaged value of n-divided respective tunnel current measurement values. In the example illustrated in FIG. 2B, an example of n=10 is illustrated. As a feature amount characterizing a signal shape feature, 10 standardized signal feature amounts (n1, . . . , n5, . . . , n10) can be obtained by dividing a signal region into, for example, 10 in the time direction and dividing a signal averaged value in each region by the largest value of the signal (peak current intensity: Ip). The method of identifying optical isomers of a low-molecular compound by using tunnel current disclosed in the present application is a novel method found by the present inventors. Therefore, the use of a standardized amount of a time averaged value of signal n-division of an obtained waveform signal as the feature amount is also a novel discovery. Note that the feature amount described above is a preferable feature amount in implementing the identifying method but is not an essential limitation for implementing the identifying method and is an optional, additional feature. As illustrated in FIG. 3, an example of the device that implements the identifying method may include a display unit 10 for displaying necessary information in implementing the identifying method, a program memory 11 storing in advance a program used for causing the analysis unit 9 or the display unit 10 to function, and a control unit 12 for reading and executing the program stored in the program memory 11. The program may be stored in the program memory 11 in advance or may be stored in a storage medium and then stored in the program memory 11 by using installation means.

For the display unit 10, a known display device such as a liquid crystal display, a plasma display, an organic EL display, or the like can be used.

The identifying method may include, subsequent to the analysis step, a ratio calculation step of calculating the ratio of D-isomers and L-isomers of a low-molecular compound contained in a sample solution. The identifying method disclosed in the present application can identify from the measurement result of the measured tunnel current whether each low-molecular compound that has passed between the pair of measuring electrodes 4a and 4b is of a D-isomer or an L-isomer. Therefore, the ratio of D-isomers and L-isomers of the low-molecular compound contained in the sample solution can be calculated based on the analysis result of the analysis step.

Note that the identifying method disclosed in the present application is useful for identifying a low-molecular compound having optical isomers as described above but can be used for identifying a low-molecular compound having no optical isomer. For example, when the sample solution is a mixed solution of glycine having no optical isomer and an amino acid having optical isomers, the glycine can be identified, and whether the amino acid other than the glycine is of a D-isomer or an L-isomer can be identified.

The identifying method disclosed in the present application achieves the following advantageous effects.
(1) when a compound having asymmetric atoms is chemically synthesized, D-isomers and L-isomers are present in a mixed manner, and D-isomers may adversely affect human bodies. Since the use of the identifying method disclosed in the present application makes it possible to identify whether the low-molecular compound is of a D-isomer or an L-isomer, safety is improved when a chemically synthesized low-molecular compound is used for pharmaceuticals or foods.
(2) For amino acids, L-isomers are used in a substantial majority in biology. Contrarily, in most non-biological processes, L-isomers and D-isomers are generated in substantially the same quantity. Therefore, enantiomer (optical isomer) excess of biologically related molecules is proposed as a biosignature in the search for extraterrestrial life. Since the use of the identifying method disclosed in the present application makes it possible to examine the ratio of D-isomers and L-isomers of a low-molecular compound contained in a sample, the identifying method disclosed in the present application can be utilized in the search for extraterrestrial life.
(3) The identifying method disclosed in the present application makes it possible to identify whether a low-molecular compound is of a D-isomer or an L-isomer by causing the low-molecular compound to flow through a microscopic gap between a pair of measuring electrodes. Therefore, the time required for identification can be reduced. Further, the size of the device can be significantly reduced compared to the conventional one.
(4) Since a D-isomer and an L-isomer have the same physical properties, identification between the D-isomer and the L-isomer is performed relying on a difference in optical rotation. However, the identifying method relying on a difference in optical rotation is to identify the whole sample and is unable to identify whether a low-molecular compound is of a D-isomer or an L-isomer on a single molecule basis. In contrast, the identifying method disclosed in the present application can identify the type of a low-molecular compound on a single molecular basis and identifying whether the low-molecular compound is of a D-isomer or an L-isomer. It is therefore possible to identify the type of a low-molecular compound and, when the low-molecular compound has an optical isomer, identify whether the low-molecular compound is of a D-isomer or an L-isomer even with (a) a sample in which a D-isomer and an L-isomer of a single type of low-molecular compound are mixed, (b) a sample in which D-isomers and L-isomers of multiple types of low-molecular compounds are mixed, and also (c) a sample in which a low-molecular compound having no optical isomer is mixed to the above (a) or (b).

Although Examples are presented below to specifically describe the disclosed details of the present application, these Examples are provided for reference of specific forms. These exemplary illustrations are intended to neither limit nor restrict the scope of the disclosure in the present application.

### EXAMPLES

### <Production of Device 1>

The device 1 was produced in accordance with the following procedure.
(1) An insulating layer was formed with polyimide on a silicon substrate.
(2) A metal layer used for forming measuring electrodes on the insulating layer was deposited on the insulating layer by using electron beam lithography and liftoff technology. ZEP520A was used for the resist, and gold was used for the material of the metal layer for forming the measuring electrodes.
(3) A deposition layer of SiO₂ was formed by chemical vapor deposition. A resist layer was laminated on the deposition layer by spin coating. ZEP520A was used for the resist.
(4) Patterns of channels including the measurement channel and patterns of pillars were formed by electron beam lithography so that these patterns were overlapped with the metal layer used for forming the measuring electrodes.
(5) The channels and pillars were formed by dry etching. A gap (nanogap G) was then formed in the metal layer by bending the substrate by MCBJ, and thus the measuring electrodes were formed. Further, to activate (hydrophilize) the pillar surface, ozone plasma treatment was performed. Due to this treatment, double bonding between O and Si of SiO was cleaved into SiOH or SiO⁻ (the counter ion is H⁺).
(6) A cover member made of PDMS (by Dow Corning Toray Co., Ltd.) in which a supply hole for a biological sample and insertion holes for electrophoresis electrodes are formed was produced by electron beam lithography. The substrate forming the channel and the cover member were treated by ozone plasma and joined to each other. Ag/AgCl was used for the electrophoresis electrodes, which were inserted through the holes formed in the cover member.
(7) A battery was used for a power supply for electrophoresis and connected via leads to the electrophoresis electrodes of the produced device. In a tunnel current detection unit, a method of amplifying current/voltage to measure a microcurrent value as a voltage and obtain a current value was applied for the ammeter, and a digital oscilloscope by National Instrument, which is an A/D converter, was used as the voltmeter. Further, a feedback resistor was incorporated into a commercially available current amplifier to increase the accuracy of the current amplifier. Further, data obtained by measuring known amino acids in advance, which will be described later, was stored in a memory of a computer. Further, a program that causes the computer to function to perform an autonomous analysis step of the identifying method was created and stored in the memory.

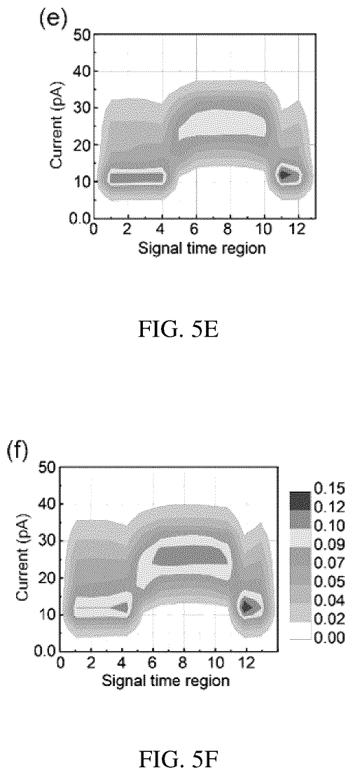

FIG. 4 represents a photograph of the produced device 1 and an enlarged photograph of a part near the measurement channel 32 in which the measuring electrodes 4 are arranged. The gap between the pair of measuring electrodes 4a and 4b was 0.56 nm.

### <Procedure to Perform Identifying Method>

### (1) Sample

### (1-1) Sample Having Optical Isomer

A total of 19 types of amino acids, namely, alanine, arginine, asparagine, aspartic acid, cysteine, glutamine, glutamic acid, histidine, isoleucine, leucine, lysine, methionine, phenylalanine, proline, serine, threonine, tryptophan, tyrosine, and valine were used. For the 19 types of amino acids, D-isomers and L-isomers were used, respectively.

### (1-2) Sample Having No Optical Isomer

Glycine was used.

The amino acids listed above were purchased from Merck Sharp & Dohme K. K., Japan.

### (2) Adjustment of Sample Solution

The samples in (1) listed above were dissolved in a phosphate buffer (10×PBS, FUJIFILM Wako Pure Chemical Corporation, Japan) to be 0.1 mM.

### (3) Measurement Step

A sample solution was supplied into the sample solution supply channel 31, and ultrapure water was supplied into the collection channel 34. DC voltages 600 mV and −600 mV were applied to the electrophoresis electrodes 61 and 62, respectively. A DC voltage of 100 mV was applied so as to span the measuring electrodes 4a and 4b. In a state of electrophoresis, a change in tunnel current occurring when the amino acid contained in the sample solution passes between the measuring electrodes 4a and 4b was measured over time.

### (4) Analysis Step

The waveform of tunnel current of (3) described above was compared with the data stored in the memory and subjected to base call. Note that machine learning was used for this comparison. Python 3.9.7 was used for the machine learning, and a scikit-learn library, version 0.24.2 was used for a classifier. Detection signals were trained and tested by using supervised machine learning using an XGBoost classifier of the scikit-learn library, version 0.24.2. A 10-fold cross-validation was performed to divide all the data of a single-molecule signal into 10 sub-datasets. Then, 10 classes of classification were performed on each of sub-datasets to be used as test data, and the remaining sub-datasets were used as training data. The average ratio of 10 classes of classification is represented in a confusion matrix. Note that, for feature amounts, “standardized amount of a time averaged value of signal n-division (n=10)” described above, “signal standard deviation”, and “signal absolute value from the signal baseline” were used. Note that “signal standard deviation” means a value found by subtracting averaged value xa of all the waveform data points from each data point of waveform data consisting of data points x1, x2, x3, . . . , xn to have ((x1−xa), (x2−xa), (x3−xa), . . . , (xn−xa)), thereby finding a deviation, finding the variance through the root mean square of the deviation, and taking positive square root of the variance. This serves as an index of variation of signals and thus represents passage behavior induced by the conformation of a molecular. Further, for “signal absolute value from the signal baseline”, first, the signal baseline is defined as the most frequent value in a certain region of signals (for example, the most frequent value out of 2000 data points) xb. The signal peak value indicates a relative signal intensity considered as an increased value of the current value from the baseline (xb). The “signal absolute amount from the signal baseline” indicates a current value of an observed signal peak value.

### Example 1: Identification of L-Isomer and D-Isomer of Individual Amino Acid

An experiment was performed to know whether or not an L-isomer and a D-isomer can be identified for individual amino acids in accordance with <Procedure to perform identifying method> described above.

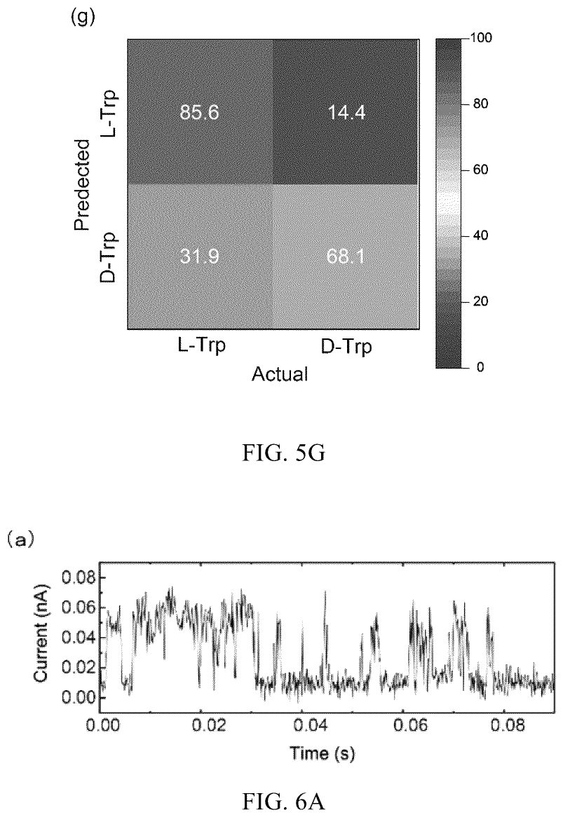

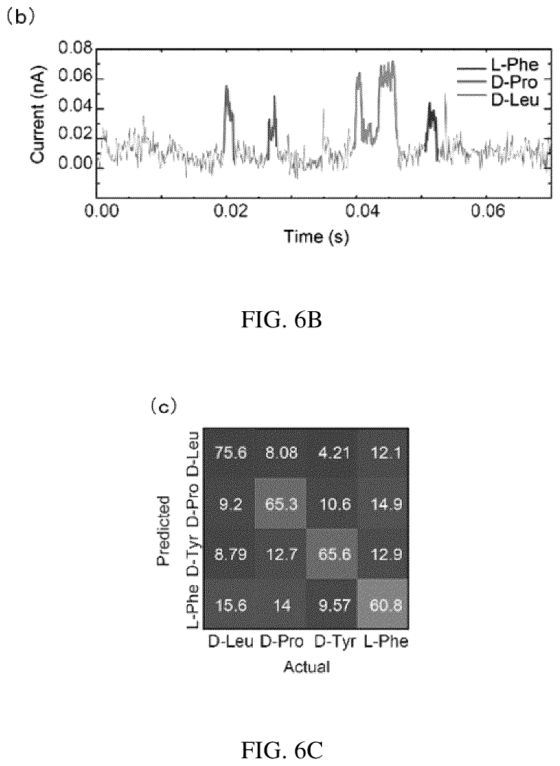

FIG. 5A illustrates analysis results when an L-isomer tryptophan and a D-isomer tryptophan were used as amino acids and each measured separately. Graph (a) illustrates a waveform signal of the L-tryptophan measured in the measurement step. Graph (b) illustrates a waveform signal of the D-tryptophan measured in the measurement step. Graph (c) enlarges a part of the waveform signal of graph (a) and illustrates a height (Ip) and a passage duration (td) of a change in the current value obtained when a single L-tryptophan passes between the pair of the measuring electrodes 4a and 4b. Graph (d) illustrates a histogram of 25143 waveform signals obtained in graph (a) and 46129 waveform signals obtained in graph (b). Note that, in the histogram illustrated in graph (d), L-Trp and D-Trp are illustrated alternatingly in the order of the leftmost of L-Trp and followed by D-Trp, L-Trp, D-Trp. Graph (e) represents a heat map of the conductance of each signal time region of L-tryptophan, and graph (f) represents a heat map of the conductance of each signal time region of D-tryptophan. FIG. 5B (g) illustrates a confusion matrix of L-tryptophan and D-tryptophan, and respective matrix quadrant values represent true positive and false positive.

As illustrated in FIG. 5A (d), the Ip histogram of L-tryptophan exhibited the largest value at 35.7 pA, and the Ip histogram of D-tryptophan exhibited the largest value at 36.9 pA. Note that, although depiction is omitted, the td histogram of L-tryptophan exhibited the largest value at 3.55 ms, and the td histogram of D-tryptophan exhibited the largest value at 3.44 ms. Although the waveform signals appear to be overlapped, the values Ip and td are a part of information included in the waveform signals. On the other hand, in FIG. 5A (e) that is a complete heat map of the values Ip and td, the waveform of L-tryptophan is wider than the waveform of D-tryptophan, which exhibited a significant difference. The standard deviation was 17.6 to 21.6 pA for L-tryptophan and 16.9 to 26.1 pA for D-tryptophan. It was confirmed from the above results that L-isomers and D-isomers can be identified by analysis of waveform signals of tryptophan.

Further, since the recall and the precision calculated from the result illustrated in FIG. 5B were 0.73 and 0.856, respectively, the F-measure was 0.798. Since the F-measure projected in random identification is 0.5 (½), it was confirmed that the identifying method disclosed in the present application makes it possible to identify D-isomers and L-isomers of tryptophan at high accuracy.

Next, for amino acids having optical isomers other than tryptophan, the F-measure was found in the same procedure as for the above tryptophan. Table 1 lists F-measures and the averaged value of a total of 19 types of amino acids including tryptophan.

TABLE 1

| Amino acid | 3-letter symbol | F-measure |
|---|---|---|
| Alanine | Ala | 0.925 |
| Arginine | Arg | 0.948 |
| Asparagine | Asn | 0.836 |
| Aspartate | Asp | 0.894 |
| Cysteine | Cys | 0.852 |
| Glutamine | Gln | 0.757 |
| Glutamate | Glu | 0.909 |
| Histidine | His | 0.897 |
| Isoleucine | Ile | 0.972 |
| Leucine | Leu | 0.952 |
| Lysine | Lys | 0.795 |
| Methionine | Met | 0.943 |
| Phenylalanine | Phe | 0.875 |
| Proline | Pro | 0.973 |
| Serine | Ser | 0.849 |
| Threonine | Thr | 0.924 |
| Tryptophan | Trp | 0.846 |
| Tyrosine | Tyr | 0.868 |
| Valine | Val | 0.875 |
| Average | — | 0.889 |

As is clear from Table 1, the F-measure was 0.757 even for glutamine having the lowest value of the F-measure, and the average was 0.889. Since the F-measure when L-isomers and D-isomers are projected by random identification is 0.5 (½), it was confirmed that L-isomers and D-isomers can be identified at high accuracy by the identifying method disclosed in the present application.

### Example 2: Identification of L-Isomer and D-Isomer in Amino Acid Mixed Solution

An experiment was performed in the same procedure as in Example 1 except that an amino acid mixed solution having a molar mixing ratio of D-Leu:D-Pro:D-Tyr:L-Phe=1:1:1:1 was used as a sample.

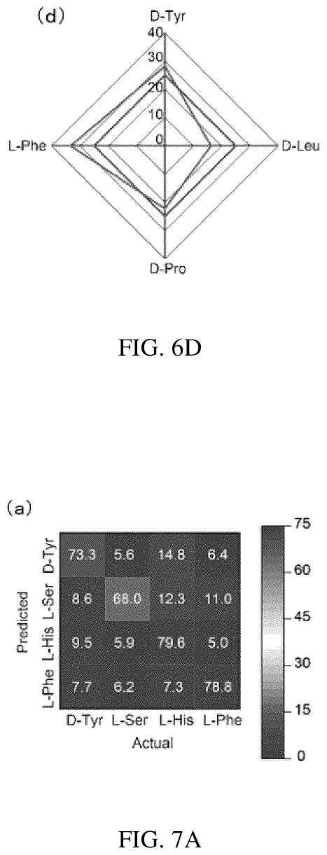

FIG. 6 illustrates the result. FIG. 6(a) illustrates a waveform signal of mixed amino acids measured in the measurement step, and FIG. 6(b) illustrates a waveform signal enlarged from the waveform signal of FIG. 6(a). FIG. 6(c) illustrates a result of a confusion matrix of D-Leu, D-Pro, D-Tyr, and L-Phe. FIG. 6(d) is a graph illustrating comparison between estimated count signals.

In the analysis step, 3800 extracted waveform signals were subjected to machine learning, and four amino acids were identified at F-measure=0.658 (FIG. 6(c)). The numbers of waveform signals identified as L-Phe, D-Leu, D-Pro, and D-Tyr were 12651, 6189, 8443, and 10721, respectively. The ratio of L-Phe, D-Leu, D-Pro, and D-Tyr to all the waveform signals were 33.3%, 16.3%, 22.2%, and 28.2%, respectively (FIG. 6(d)). The errors in identifying amino acids were 11.3%, 5.6%, 7.6%, and 9.6%, respectively. From the above result, the existence ratios of L-Phe, D-Leu, D-Pro, and D-Tyr in the amino acid mixed solution were estimated to be 33.3±11.3%, 16.3±5.6%, 22.2±7.6%, and 28.2±9.6%, respectively (see Table 2).

### Example 3: Identification of L-Isomer and D-Isomer in Amino Acid Mixed Solution

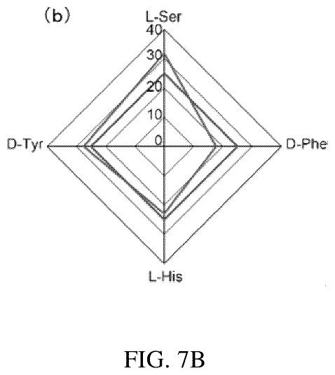

An experiment was performed in the same procedure as in Example 2 except that L-Phe, L-His, L-Ser, and D-Tyr were used at an equal molar ratio as the amino acid mixed solution. FIG. 7 illustrates the result. FIG. 7(a) illustrates a result of a confusion matrix of L-Phe, L-His, L-Ser, and D-Tyr. FIG. 7(b) is a graph illustrating comparison between estimated count signals.

Four amino acids were identified at F-measure=0.748 (FIG. 7(a)). The existence ratios of L-Phe, L-His, L-Ser, and D-Tyr were estimated to be 17.7±4.2%, 23.0±5.6%, 31.8±7.7%, and 27.5±6.6%, respectively (FIG. 7(b) and Table 2).

TABLE 2

| | Amino | | Estimated |
|---|---|---|---|
| Sample name | acid | F-measure | Radio (%) |
| L-Phe:D-Leu:D- | L-Phe | 0.658 | 33.3 ± 11.3 |
| Pro:D-Tyr = | D-Leu | | 16.3 ± 5.6 |
| 1:1:1:1 | D-Pro | | 22.2 ± 7.6 |
| | D-Tyr | | 28.2 ± 9.6 |
| D-Tyr:L-Ser:L- | D-Tyr | 0.748 | 27.5 ± 6.6 |
| His:L-Phe = | L-Ser | | 31.8 ± 7.7 |
| 1:1:1:1 | L-His | | 23.0 ± 5.6 |
| | L-Phe | | 17.7 ± 4.2 |

### Example 4: Identification of Glycine in Amino Acid Mixed Solution

An experiment was performed in the same procedure as in Example 2 except that D-Trp, D-Thr, D-Asn, L-Met, and Gly were used at an equal molar ratio (the ratio of each amino acid is 20%) as the amino acid mixed solution. Table 3 lists the result. Five amino acids were identified at F-measure=0.808. The existence ratios of D-Trp, D-Thr, D-Asn, L-Met, and Gly were estimated to be 13.6±6.5%, 19.8±7.8%, 18.26±5.3%, 19.54±4.3%, and 28.8±9.8%, respectively.

TABLE 3

| | Amino | | Estimated |
|---|---|---|---|
| Sample name | acid | F-measure | Ratio (%) |
| DTrp:DThr:DAsn:LMet:Gly = | DTrp | 0.808 | 13.6 ± 6.5 |
| 1:1:1:1:1 | DThr | | 19.8 ± 7.8 |
| | DAsn | | 18.26 ± 5.3 |
| | LMet | | 19.45 ± 4.3 |
| | Gly | | 28.8 ± 9.8 |

From the results of Example 2 and Example 3, the amino acid solution in which D-isomers and L-isomers were mixed was identified at a high F-measure. Therefore, it was confirmed that, for the low-molecular compound in which D-isomers and L-isomers are mixed, the type of the low-molecular compound and whether the low-molecular compound is of a D-isomer or an L-isomer can be identified at high accuracy by the identifying method disclosed in the present application. Further, from the result of Example 4, even when glycine having no optical isomer is further mixed to an amino acid solution in which D-isomers and L-isomers are mixed, glycine was identified at high accuracy. Therefore, it was confirmed that a low-molecular compound having no optical isomer can be identified together with identification of D-isomers and L-isomers by the identifying method disclosed in the present application.

### Example 5: Alcohol Molecule

Next, an experiment to identify D-isomers and L-isomers of a compound other than amino acids was performed.

### (1) Sample

The following two sets, namely, a total of four types of alcohol molecules were used. The alcohol molecules were purchased from FUJIFILM Wako Pure Chemical Corporation (Tokyo, Japan).

### <1-Phenyl-1-Butanol>

SP: (S)-1-Phenyl-1-butanol
RP: (R)-1-Phenyl-1-butanol

### <1-Naphtyl-Ethanol>

SN: (S)-1-Naphtyl-ethanol
RN: (R)-1-Naphtyl-ethanol

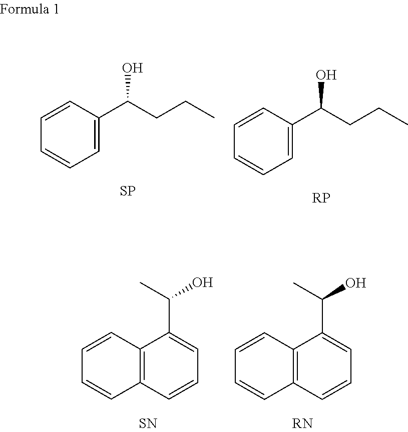

### (2) Adjustment of Sample Solution

The purchased alcohol molecules were used as they were without being subjected to further refinement. The alcohol molecules were dissolved in Milli-Q water and adjusted to 100 nM.

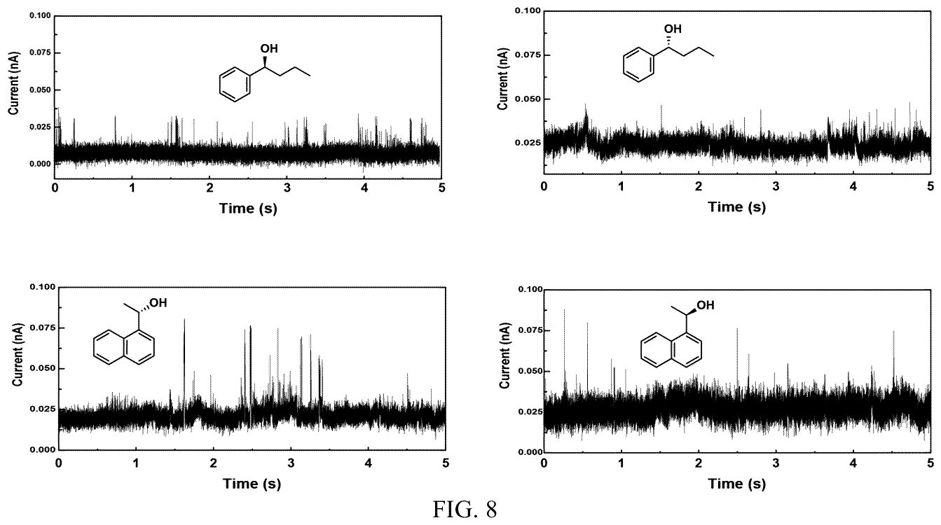

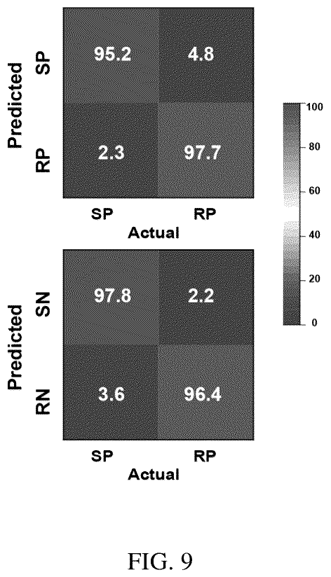

Identification of respective alcohols was performed in accordance with the above <Procedure to perform identifying method> except that the sample was changed to the alcohol molecule and adjusted as described above. FIG. 8 illustrates waveform signals of four types of alcohol molecules measured in the measurement step. As a result of 15-minute measurement, 288 single-molecule signals were obtained for SN, so were 210 for RN, 843 for SP, and 1178 for RP. For each molecular signal, machine learning was performed on 80% of the molecular signals as training data, and the remaining 20% was used as test data to perform evaluation of a learning machine. The evaluation of identifying capacity was performed at F1-score (F-measure). FIG. 9 illustrates the results. As illustrated in FIG. 9, the identification capacity for S-isomers and R-isomers of 1-Phenyl-1-butanol was 96.2%, and the identification capacity for S-isomers and R-isomers of 1-Naphtyl-ethanol was 97%.

### Example 6: Collected Sand

As one of the actual usage forms of the identifying method disclosed in the present application, identification of the presence or absence of an organism in sand mined from the ground is assumed. In such a case, conceivable molecules passing between the measuring electrodes 4 may be amino acids, nucleic acids, or sugars. Herein, a case where guanosine (dGMP) and thymine (dTMP), which are nucleic acid monomers, exist as impurities is assumed. Accordingly, the single-molecule identification was performed on seven types of: dGMP; dTMP; L-isomers and D-isomers of alanine (Ala) and histidine (His); and glycine (Gly) having no optical isomer.

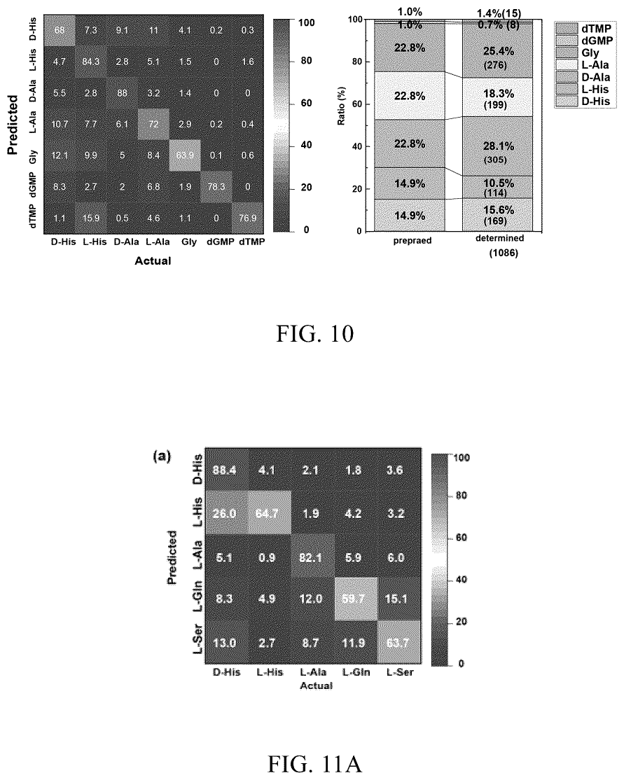

All the samples were purchased from FUJIFILM Wako Pure Chemical Corporation (Tokyo, Japan), and measurement was performed in accordance with the above <Procedure to perform identifying method>. FIG. 10 illustrates the measurement results. In the right graph of FIG. 10, “Prepared” represents a mixture ratio of amino acids and nucleic acids mixed in advance with D-His, L-His, D-Ala, L-Ala, Gly, dGMP, and dTMP being placed in this order from the bottom.

The accuracy in identifying seven types (nucleic acid:dGMP, dTMP; amino acid: L-Ala, D-Ala, L-His, D-His, Gly) calculated from the results illustrated in FIG. 10 was F-measure=0.761. Since the random identification accuracy is 0.143 (= 1/7), it is confirmed that seven types of mixture molecules were accurately identified in Example 6.

### Example 7: Quantitative Analysis 1

Next, quantitative analysis was performed on a mixed aqueous solution mixed with various amino acids. Five types of amino acids of D-His, L-His, L-Ala, L-Gln, and L-Ser were used for samples, and aqueous solutions in which respective amino acids were mixed at an equal molar number were prepared. The F-measure based on a single current-time waveform (a single signal) was 0.72. Next, a mixed aqueous solution in which D-His, L-His, L-Ala, L-Gln, and L-Ser were mixed was prepared. The concentration of total amino acids of the mixed aqueous solution was 100 nM, and D-His, L-His, L-Ala, L-Gln, and L-Ser were mixed at the following mixture ratios.
(1) 1:1:1:1:1
(2) 3:1:1.5:1.5:1.5

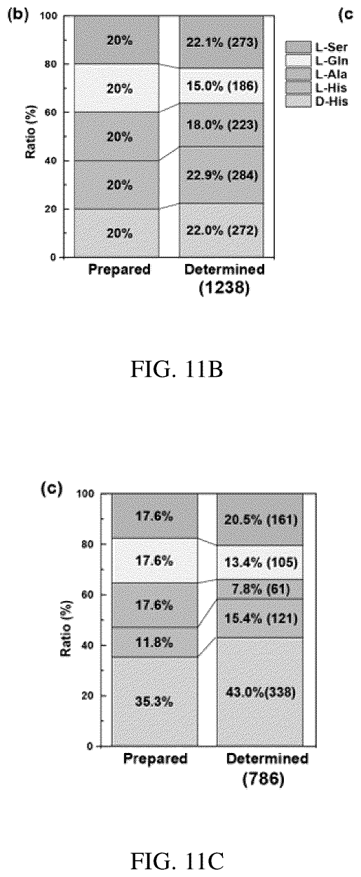

Next, measurement was performed on the mixed aqueous solutions at the mixture ratios of (1) and (2) described above in accordance with the above <Procedure to perform identifying method>. FIG. 11 illustrates the measurement results. In FIG. 11(b), “Prepared” represents the mixture ratio of the amino acids mixed at the ratio of (1) described above, and in FIG. 11(c), “Prepared” represents the mixture ratio of the amino acids mixed at the ratio of (2) described above with D-His, L-His, L-Ala, L-Gln, and L-Ser being placed in this order from the bottom.

Then, 1238 and 768 current-time waveforms obtained by the measurement were classified into D-His, L-His, L-Ala, L-Gln, and L-Ser by machine learning. In FIG. 11(b) and(c), “Determined” represents the classified result. The ratios of the counts of the number of classified waveforms were as follows.
The mixed aqueous solution of (1)→1:1.04:0.82:0.68:1.00
The mixed aqueous solution of (2)→3:1.07:0.54:0.93:1.43

As is clear from FIGS. 11(b) and(c), it was confirmed that the use of identifying method disclosed in the present application makes it possible to accurately identify amino acids in the mixed aqueous solution in which D-isomers and L-isomers are mixed.

### Example 8: Quantitative Analysis 2

Next, quantitative analysis was performed using a mixed aqueous solution with changed mixture ratios of D-His and L-His. The concentration of total amino acids of the mixed aqueous solution was 100 nM, and D-His and L-His were mixed at the following mixture ratios.
(1) 1:1
(2) 1:2
(3) 1:3

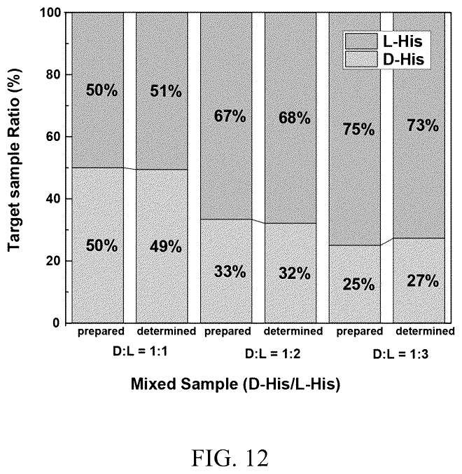

Next, measurement was performed on the mixed aqueous solution at the mixture ratios of (1) to (3) described above in accordance with the above <Procedure to perform identifying method>. FIG. 12 illustrates the measurement result. In FIG. 12, “Prepared” represents the mixture ratios of the amino acids mixed at the ratios of (1) to (3) described above with D-His and L-His being placed in this order from the bottom.

The current-time waveforms obtained by the measurement were classified into D-His and L-His by machine learning. The label “Determined” represents the classified result. The ratios of the counts of the number of classified waveforms were as follows.
The mixed aqueous solution of (1)→1:1.0
The mixed aqueous solution of (2)→1:2.1
The mixed aqueous solution of (3)→1:2.7
The error was less than or equal to 12% for all the mixture ratios.

From the above results, it was confirmed that the use of the identifying method disclosed in the present application makes it possible to accurately identify various samples in mixed aqueous solutions in which D-isomers and L-isomers are mixed.

### INDUSTRIAL APPLICABILITY

The use of the identifying method of optical isomers of a low-molecular compound disclosed in the present application makes it possible to identify whether each measured low-molecular compound is of a D-isomer or an L-isomer. Therefore, the identifying method disclosed in the present application is useful in the pharmaceutical industry and the food industry.

### LIST OF REFERENCE SYMBOLS

1 device
2 substrate
3 channel
31 sample solution supply channel
32 measurement channel
33 first tapered channel
34 collection channel
35 second tapered channel
4, 4a, 4b measuring electrode
6, 6a, 6b power supply for electrophoresis
61 electrophoresis first electrode
62 electrophoresis second electrode
7 tunnel current detection unit
8 power supply for tunnel current measurement
9 analysis unit
10 display unit
11 program memory
12 control unit

1. An identifying method of optical isomers of a low-molecular compound, wherein the identifying method is performed by using a device comprising measuring electrodes for measuring tunnel current occurring when the low-molecular compound passes between the measuring electrodes, the identifying method comprising:
a low-molecular compound electrophoresis step of applying a voltage so as to span the measuring electrodes of the device to cause a low-molecular compound contained in a sample solution to pass between the measuring electrodes by electrophoresis;
a measurement step of measuring tunnel current occurring when the low-molecular compound passes through a gap between the measuring electrodes; and
an analysis step of analyzing from the measured tunnel current whether each measured low-molecular compound is of a D-isomer or an L-isomer.

2. The identifying method according to claim 1 further comprising, subsequent to the analysis step, a ratio calculation step of calculating a ratio of D-isomers and L-isomers of the low-molecular compound contained in the sample solution.

3. The identifying method according to claim 1, wherein the low-molecular compound is an amino acid, and the amino acid is of one or more types selected from a group consisting of alanine, arginine, asparagine, aspartic acid, cysteine, glutamine, glutamic acid, histidine, isoleucine, leucine, lysine, methionine, phenylalanine, proline, serine, threonine, tryptophan, tyrosine, and valine.

4. The identifying method according to claim 1, wherein the measuring electrodes are not modified with a substance that interacts with the low-molecular compound.

5. The identifying method according to claim 3, wherein the measuring electrodes are not modified with a substance that interacts with the low-molecular compound.

6. The identifying method according to claim 2, wherein the low-molecular compound is an amino acid, and the amino acid is of one or more types selected from a group consisting of alanine, arginine, asparagine, aspartic acid, cysteine, glutamine, glutamic acid, histidine, isoleucine, leucine, lysine, methionine, phenylalanine, proline, serine, threonine, tryptophan, tyrosine, and valine.

7. The identifying method according to claim 2, wherein the measuring electrodes are not modified with a substance that interacts with the low-molecular compound.

8. The identifying method according to claim 6, wherein the measuring electrodes are not modified with a substance that interacts with the low-molecular compound.
